SEQUENCE LISTING (2 sequences)

SEQ ID NO: 1 - DNA, 515 bp, Homo sapiens
  CDS (26)..(436)
acggctgcccaacccggtcccagccatgtccaacgtcccccacaagtcctcactgcccgagggcatccgccctggcacggtgctgagaattcgcggcttggttcctcccaatgccagcaggttccatgtaaacctgctgtgcggggaggagcagggctccgatgccgcgctgcatttcaacccccggctggacacgtcggaggtggtcttcaacagcaaggagcaaggctcctggggccgcgaggagcgcgggccgggcgttcctttccagcgcgggcagcccttcgaggtgctcatcatcgcgtcagacgacggcttcaaggccgtggttggggacgcccagtaccaccacttccgccaccgcctgccgctggcgcgcgtgcgcctggtggaggtgggcggggacgtgcagctggactccgtgaggatcttctgagcagaagcccaggcgggcccggggccttggctggcaaataaagcgttagcccgcagcgaaaaaaaaaaaaaaaaaaaaa

SEQ ID NO: 2 - protein, 136 aa, Homo sapiens
MSNVPHKSSLPEGIRPGTVLRIRGLVPPNASRFHVNLLCGEEQGSDAALHFNPRLDTSEVVFNSKEQGSWGREERGPGVPFQRGQPFEVLIIASDDGFKAVVGDAQYHHFRHRLPLARVRLVEVGGDVQLDSVRIF

(19) United States
(12) Patent Application Publication
(10) Pub. No.: US 2015/0330985 A1
(43) Pub. Date: Nov. 19, 2015

(54) GALECTIN-7 AS A BIOMARKER FOR DIAGNOSIS, PROGNOSIS AND MONITORING OF OVARIAN AND RECTAL CANCER

(71) Applicant: INSTITUT NATIONAL DE LA RECHERCHE SCIENTIFIQUE, Québec (CA)

(72) Inventor: Yves St-Pierre, Laval (CA)

(21) Appl. No.: 14/654,195

(22) PCT Filed: Dec. 20, 2012
(86) PCT No.: PCT/CA2012/050924

§ 371 Date: Jun. 19, 2015

(51) Int. Cl. G01N 33/574; G06F 19/00

(52) U.S. Cl. CPC G01N 33/57449; G01N 33/57419; G06F 19/345; G01N 2333/4703

(57) ABSTRACT

Methods, kits and systems for the diagnosis, prognosis and monitoring of ovarian cancer and rectal cancer are described. The methods, kits and systems are based on the detection of the lectin Galectin-7 in samples obtained from subjects.

### FIELD OF THE INVENTION

The present invention relates to the diagnosis and prognosis of cancer, and more particularly to the diagnosis and prognosis of ovarian and rectal cancer.

### BACKGROUND OF THE INVENTION

Cancer is a generic term for a large group of diseases that can affect any part of the body. There are over 200 different types of cancer because there are over 200 different types of body cells. Most cancers, however, originate from transformation of epithelial cells. In fact, cancers of the epithelial cells make up about 85% of all cancers. Given the heterogeneity of epithelial cancers, there is a clear clinical need to identify predictive markers and novel treatments that will improve patient treatment.

Ovarian cancer is the fifth leading cause of cancer-related deaths in the Western world, the second most common gynecological cancer and the leading cause of death from gynecological malignancies. They are generally classified histologically as serous, endometrioid, mucinous, clear cell, as well as other less common types. Over 90% of ovarian cancers are of epithelial origin. In the United States, epithelial ovarian cancer is the leading cause of gynecologic cancer death and the fifth most common cause of cancer mortality among women. Worldwide, nearly 200,000 new cases and more than 125,000 deaths are attributable to the disease each year. The majority of patients are diagnosed with advanced disease, for which the standard treatment is aggressive surgical debulking followed by platinum-based chemotherapy. Because of high toxicity and the absence of reliable biomarkers, a high percentage of patients are unable to complete therapy or die within a few years. It is thus important to develop biomarkers that are useful in stratifying advanced-stage ovarian cancer patients to identify patients with worse predicted outcomes and redirect them to appropriate and optimal treatments.

According to the National Cancer Institute, in 2012, nearly 150,000 new cases and more than 50,000 deaths will be attributable to cancer of the colon and rectum in the United States. For both cancers, symptoms may include gastrointestinal bleeding, change in bowel habits, abdominal pain, intestinal obstruction, weight loss, and weakness. Although colon and rectal cancer are often epidemiologically related, (i.e., colorectal cancer), rectal cancer refers to tumors that arise within 15 centimeters from the anal sphincter. Accurate staging provides crucial information about the location and size of the primary tumor in the rectum, and, if present, the size, number, and location of any metastases. In the case of rectal cancer, physical examination may also reveal a palpable mass and bright blood in the rectum. Accurate staging will help to determine the type of surgical intervention and the choice of therapy. Initial staging procedures may include digital-rectal examination and/or rectovaginal exam and rigid proctoscopy, colonoscopy, pan-body computed tomography (CT) scan magnetic resonance imaging (MRI) of the abdomen and pelvis, endorectal ultrasound (ERUS), and positron emission tomography (PET) for prognostic assessment. Local resection strategies will depend on our ability to predict the extent of pathological response using clinical, molecular, and imaging biomarkers.

It is important to individualize rectal cancer treatment. For example, in some cases, patients may need an intensified regimen to increase tumor response, whereas others may be treated using only standard chemoradiotherapy. Accordingly, reliable clinical biomarkers are needed for accurate stratification to apply therapeutic options with high certainty. There is also a clinical need for biomarkers that could be used pre-therapeutically to predict the response of an individual patient's tumor to multimodal treatment and that could be implemented into clinical decision-making. For example, patients with a biomarker profile indicating “responder to standard treatment” would be subjected to a low-toxicity preoperative regimen whereas patients with a biomarker profile indicating “nonresponder to standard treatment,” would be subjected to a more aggressive approach.

The present description refers to a number of documents, the content of which is herein incorporated by reference in their entirety.

### SUMMARY OF THE INVENTION

The present invention relates to the diagnosis and prognosis of cancer, and more particularly to the diagnosis and prognosis of ovarian and rectal cancer.

More specifically, in accordance with the present invention, there is provided a method for determining whether a subject has ovarian cancer or a predisposition to develop ovarian cancer, said method comprising: measuring the level of expression of Galectin-7 in an ovarian cell and/or tissue sample from said subject; comparing said level of expression to a control level; and determining whether said subject has ovarian cancer or a predisposition to develop ovarian cancer based on said comparison.

In an embodiment, the above-mentioned control level is a level measured in a non-cancerous ovarian cell and/or tissue sample, and (i) a higher level of expression in the ovarian cell and/or tissue sample from said subject is indicative that said subject has ovarian cancer or a predisposition to develop ovarian cancer; or (ii) a similar or lower level of expression in the ovarian cell and/or tissue sample from said subject is indicative that said subject does not have ovarian cancer or a predisposition to develop ovarian cancer.

In another embodiment, the above-mentioned control level is a level measured in a cancerous ovarian cell and/or tissue sample, and (i) a similar or higher level of expression in the ovarian cell and/or tissue sample from said subject is indicative that said subject has ovarian cancer or a predisposition to develop ovarian cancer; or (ii) a lower level of expression in the ovarian cell and/or tissue sample from said subject is indicative that said subject does not have ovarian cancer or a predisposition to develop ovarian cancer.

In an aspect, the present invention provides a method for determining whether a subject has rectal cancer or a predisposition to develop rectal cancer, said method comprising: measuring the level of expression of Galectin-7 in a rectal cell and/or tissue sample from said subject; comparing said level of expression to a control level; and determining whether said subject has rectal cancer or a predisposition to develop rectal cancer based on said comparison.

In an embodiment, the above-mentioned control level is a level measured in a non-cancerous rectal cell and/or tissue sample, and (i) a higher level of expression in the rectal cell and/or tissue sample from said subject is indicative that said subject has rectal cancer or a predisposition to develop rectal cancer; or (ii) a similar or lower level of expression in the rectal cell and/or tissue sample from said subject is indicative that said subject does not have rectal cancer or a predisposition to develop rectal cancer.

In another embodiment, the above-mentioned control level is a level measured in a cancerous rectal cell and/or tissue sample, and (i) a similar or higher level of expression in the rectal cell and/or tissue sample from said subject is indicative that said subject has rectal cancer or a predisposition to develop rectal cancer; or (ii) a lower level of expression in the rectal cell and/or tissue sample from said subject is indicative that said subject does not have rectal cancer or a predisposition to develop rectal cancer.

In another aspect, the present invention provides a method for monitoring the progression of ovarian or rectal cancer in a subject, the method comprising: measuring the level of expression of Galectin-7 in a first ovarian or rectal cell and/or tissue sample from said subject at a first time point; measuring the level of expression of Galectin-7 in a second ovarian or rectal cell and/or tissue sample from said subject at a later time point; wherein (a) a level of expression of Galectin-7 that is higher in said second sample relative to said first sample is indicative that said ovarian or rectal cancer has progressed; (b) a level of expression of Galectin-7 that is lower in said second sample relative to said first sample is indicative that said ovarian or rectal cancer has regressed; or (c) a level of expression of Galectin-7 that is similar in said second sample relative to said first sample is indicative that said ovarian or rectal cancer is stable. In an embodiment, the subject is undergoing anti-cancer therapy between said first time point and said later time point.

In another aspect, the present invention provides a method for monitoring the progression of ovarian or rectal cancer in a subject, the method comprising: measuring the level of expression of Galectin-7 in an ovarian or rectal cell and/or tissue sample (a “later” sample) from said subject, and comparing said level of expression to an earlier level of expression determined in an ovarian or rectal cell and/or tissue sample from said subject obtained at an earlier time; wherein (a) a level of expression of Galectin-7 that is higher in said sample relative to said earlier sample is indicative that said ovarian or rectal cancer has progressed; (b) a level of expression of Galectin-7 that is lower in said sample relative to said earlier sample is indicative that said ovarian or rectal cancer has regressed; or (c) a level of expression of Galectin-7 that is similar in said sample relative to said earlier sample is indicative that said ovarian or rectal cancer is stable. In an embodiment, the subject is undergoing anti-cancer therapy during the period between the sampling of the earlier and later samples.

In another aspect, the present invention provides a kit for diagnosing or monitoring the progression of ovarian or rectal cancer in a subject, comprising a Galectin-7 binding reagent or at least one oligonucleotide hybridizing to a Galectin-7 nucleic acid. In an embodiment, the kit further comprises instructions for using the Galectin-7 binding reagent for diagnosing and/or monitoring the progression of ovarian or rectal cancer in a subject; a labeled binding partner to the Galectin-7 binding reagent; one or more reagents; one or more containers; and/or appropriate controls/standards.

In another aspect, the present invention provides the use of a Galectin-7 binding reagent or at least one oligonucleotide hybridizing to a Galectin-7 nucleic acid for the diagnosis of ovarian or rectal cancer in a subject.

In another aspect, the present invention provides the use of a Galectin-7 binding reagent or at least one oligonucleotide hybridizing to a Galectin-7 nucleic acid for monitoring the progression of ovarian or rectal cancer in a subject.

In another aspect, the present invention provides an ovarian or rectal cancer diagnostic system comprising (i) an ovarian or rectal cell and/or tissue sample; (ii) a Galectin-7 binding reagent; and (iii) a device for detecting the presence and/or amount of Galectin-7/Galectin-7 binding reagent complexes.

In another aspect, the present invention provides a computer-readable medium comprising code for controlling one or more processors to classify whether an ovarian or rectal cell and/or tissue sample from an subject is associated with ovarian or rectal cancer, said code comprising: instructions to apply a statistical process to a data set comprising a Galectin-7 profile to produce a statistically derived decision classifying said sample as an ovarian or rectal cancer sample or non-ovarian or rectal cancer sample based upon said Galectin-7 profile, wherein said Galectin-7 profile indicates the level of Galectin-7 in said ovarian or rectal cell and/or tissue sample. In an embodiment, the computer-readable medium comprises instructions to apply a statistical process to a data set comprising said Galectin-7 profile in combination with a symptom profile which indicates the presence or severity of at least one symptom in said subject to produce a statistically derived decision classifying said sample as an ovarian or rectal cancer sample or non-ovarian or rectal cancer sample based upon said Galectin-7 profile and said symptom profile.

In another aspect, the present invention provides a system for classifying whether an ovarian or rectal cell and/or tissue sample from a subject is associated with ovarian or rectal cancer, said system comprising: (a) a data acquisition module configured to produce a data set comprising a Galectin-7 profile, wherein said Galectin-7 profile indicates the presence or level of Galectin-7 in said ovarian or rectal cell and/or tissue sample; (b) a data processing module configured to process the data set by applying a statistical process to the data set to produce a statistically derived decision classifying said sample as an ovarian or rectal cancer sample or non-ovarian or rectal cancer sample based upon said Galectin-7 profile; and (c) a display module configured to display the statistically derived decision. In an embodiment, the data processing module comprises instructions to apply a statistical process to a data set comprising said Galectin-7 profile in combination with a symptom profile which indicates the presence or severity of at least one symptom in said subject to produce a statistically derived decision classifying said sample as an ovarian or rectal cancer sample or non-ovarian or rectal cancer sample based upon said Galectin-7 profile and said symptom profile.

In an embodiment, the above-mentioned level of expression of Galectin-7 is measured at the protein level.

In a further embodiment, the level of expression of Galectin-7 is measured using a Galectin-7 binding reagent, in a further embodiment an antibody.

In an embodiment, the level of expression of Galectin-7 is measured by immunohistochemistry.

In an embodiment, the above-mentioned ovarian or rectal cell and/or tissue sample is a biopsy sample.

In an embodiment, the above-mentioned ovarian cancer is a mucinous carcinoma, a transitional cell carcinoma or an adenocarcinoma. In a further embodiment, the adenocarcinoma is endometrioid adenocarcinoma.

Other objects, advantages and features of the present invention will become more apparent upon reading of the following non-restrictive description of specific embodiments thereof, given by way of example only with reference to the accompanying drawings.

### BRIEF DESCRIPTION OF THE DRAWINGS

In the appended drawings:

FIG. 1 shows expression of Galectin-7 protein levels in normal human ovarian tissue and corresponding cancer tissues patients with ovarian cancer as determined by immunohistochemistry;

FIG. 2 is a graph showing the frequency of tissue samples obtained from patients at different stages of ovarian cancer and that showed detectable expression of Galectin-7 at the protein level as measured by immunohistochemistry;

FIGS. 3A and 3B are graphs showing a quantitative assessment of protein level of Galectin-7 in 100 cases of ovarian cancer as measured by immunohistochemistry using an ovarian disease spectrum (ovarian cancer progression) tissue microarray array, and scored using the Allred scoring system (Allred et al., Mod Pathol. 1998; 11:155-168) accounting for both the intensity of staining and the proportion of stained cells producing a sum score of the two values (intensity+proportion=0 to 8);

FIG. 4 is a graph showing the frequency of tissue samples obtained from patients with different malignant types of ovarian cancer and that showed detectable expression of Galectin-7 at the protein level as measured by immunohistochemistry;

FIG. 5 shows expression of galectin-7 protein level in normal human rectal tissue and corresponding cancer tissues patients with rectal cancer as determined by immunohistochemistry;

FIG. 6 is a graph showing the frequency of tissue samples obtained from patients at different stages of rectal cancer and that showed detectable expression of galectin-7 at the protein level as measured by immunohistochemistry;

FIG. 7 is a graph showing a quantitative assessment of protein level of galectin-7 in 63 cases of rectal cancer as measured by immunohistochemistry using a rectal disease spectrum (rectal cancer progression) tissue microarray array, and magnitudes of induction fold obtained therefrom. Scoring of tissue microarrays constructed from human rectal tissue specimens were stained with anti-galectin-7 antibody. Specimens were scored according to the Allred scoring system (Allred et al., Mod Pathol. 1998; 11:155-168) accounting for both the intensity of staining of epithelial cells and the proportion of stained cells producing a sum score of the two values (intensity+proportion=0 to 8);

FIG. 8 shows the amino acid sequence of human Galactin-7 (NCBI Reference Sequence: NP_{—}002298.1, SEQ ID NO: 2); and

FIG. 9 shows the nucleotide sequence of a nucleic acid encoding human Galactin-7 (NCBI Reference Sequence: NM_{—}002307.3; SEQ ID NO:1, coding sequence=nucleotides 26 to 436).

### DETAILED DESCRIPTION OF THE INVENTION

The present invention relates to the diagnosis and prognosis of cancer, and more particularly to the diagnosis and prognosis of ovarian and rectal cancer.

In an aspect, the present invention provides a method for determining whether a subject has ovarian cancer or a predisposition to develop ovarian cancer, said method comprising: measuring the level of expression of Galectin-7 in an ovarian cell and/or tissue sample from said subject; comparing said level of expression to a control level; and determining whether said subject has ovarian cancer or a predisposition to develop ovarian cancer based on said comparison.

In another aspect, the present invention provides a method for determining whether a subject has rectal cancer or a predisposition to develop rectal cancer, said method comprising: measuring the level of expression of Galectin-7 in an rectal cell and/or tissue sample from said subject; comparing said level of expression to a control level; and determining whether said subject has rectal cancer or a predisposition to develop rectal cancer based on said comparison.

In another aspect, the present invention provides a method for monitoring the progression of ovarian or rectal cancer in a subject, the method comprising: measuring the level of expression of Galectin-7 in a first ovarian or rectal cell and/or tissue sample from said subject at a first time point; measuring the level of expression of Galectin-7 in a second ovarian or rectal cell and/or tissue sample from said subject at a later (second) time point; wherein (i) a level of expression of Galectin-7 that is higher in said second sample relative to said first sample is indicative that said ovarian or rectal cancer has progressed; (ii) a level of expression of Galectin-7 that is lower in said second sample relative to said first sample is indicative that said ovarian or rectal cancer has regressed; or (iii) a level of expression of Galectin-7 that is similar in said second sample relative to said first sample is indicative that said ovarian or rectal cancer is stable (i.e. has not significantly progressed or regressed). In an embodiment, the method is used for treatment follow-up (for monitoring the effect of an anti-cancer treatment). In an embodiment, the subject is undergoing treatment/therapy (surgery, radiotherapy and/or chemotherapy) for the ovarian or rectal cancer between the first time point and the later time point.

In another aspect, the present invention provides a method for monitoring the progression of ovarian or rectal cancer in a subject, the method comprising: measuring the level of expression of Galectin-7 in an ovarian or rectal cell and/or tissue sample (a “later” sample) from said subject, and comparing said level of expression to an earlier level of expression determined in an ovarian or rectal cell and/or tissue sample from said subject obtained at an earlier time; wherein (a) a level of expression of Galectin-7 that is higher in said sample relative to said earlier sample is indicative that said ovarian or rectal cancer has progressed; (b) a level of expression of Galectin-7 that is lower in said sample relative to said earlier sample is indicative that said ovarian or rectal cancer has regressed; or (c) a level of expression of Galectin-7 that is similar in said sample relative to said earlier sample is indicative that said ovarian or rectal cancer is stable (i.e. has not significantly progressed or regressed). In an embodiment, the method is used for treatment follow-up (for monitoring the effect of an anti-cancer treatment). In an embodiment, the subject is undergoing treatment/therapy (surgery, radiotherapy and/or chemotherapy) during the period between the sampling of the earlier and later samples.

In another aspect, the present invention provides the use of a Galectin-7 binding reagent (e.g., an anti-Galectin-7 antibody) or at least one oligonucleotide hybridizing to a Galectin-7 nucleic acid for the diagnosis of ovarian or rectal cancer in a subject. In another aspect, the present invention provides the use of a Galectin-7 binding reagent (e.g., an anti-Galectin-7 antibody) or at least one oligonucleotide hybridizing to a Galectin-7 nucleic acid for monitoring the progression of ovarian or rectal cancer in a subject.

Galectins are a family of lectins, which are defined by a shared consensus amino acid sequence and an affinity for β-galactose-containing oligosaccharides (Liu and Rabinovitch, 2005). Galectins can be found in the cytoplasm or the nucleus or can be secreted by the cell, which occurs via a non-classical secretory pathway. The distribution of galectins is tissue specific, and their expression is developmentally regulated (Barondes et al., 1994; Kasai and Hirabayashi, 1996). The 15 members of the family are normally classified according to their structure and number of carbohydrate recognition domains (CRDs). The galectins have either one (Galectin-1, -2, -5, -7, -10, -11, -13, -14, and -15) or two (Galectin-4, -6, -8, -9, and -12) CRDs that are linked by a hinge peptide. There is also a chimeric form of galectin (i.e. galectin-3) that contains one CRD connected to a non-lectin domain.

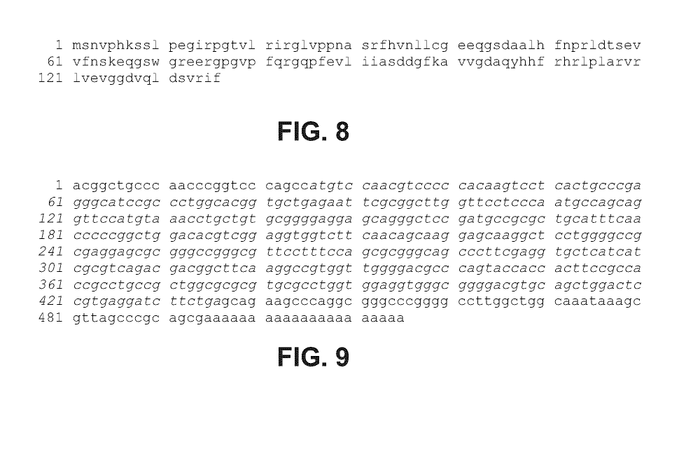

Galectin-7 was initially described by Madsen and colleagues (1995) as a marker of epithelial differentiation. Subsequent studies have confirmed that Galectin-7 is present in most normal epithelial cells, most notably stratified epithelium found in various tissues. Usually, its expression varies depending on the levels of differentiation of pluristratified epithelia, and the onset of its expression coincides with the first visible signs of epidermal stratification. The amino acid sequence of human Galectin-7 protein is depicted in FIG. 8 (SEQ ID NO: 2), and the nucleotide sequence of a human Galectin cDNA is depicted in FIG. 9 (SEQ ID NO: 1), with the coding sequence corresponding to nucleotides 26 to 346.

In view of the demonstration by the present inventor that normal (i.e. non-cancerous) ovarian and rectal tissue typically exhibit no detectable expression of Galectin-7 (see Example 2), in an embodiment the above-mentioned control level is 0 (i.e. no detectable expression), and thus the detection of any expression of Galectin-7 (i.e. irrespective of the level) in the cell and/or tissue sample from the subject is indicative that the subject has ovarian or rectal cancer or a predisposition to develop ovarian or rectal cancer.

In an embodiment, the control level is a level measured in a non-cancerous cell and/or tissue sample (a healthy tissue, an adjacent tissue), and (i) a higher level of expression in the ovarian or rectal cell and/or tissue sample from said subject is indicative that said subject has ovarian or rectal cancer or a predisposition to develop ovarian or rectal cancer; or (ii) a similar or lower level of expression in the ovarian or rectal cell and/or tissue sample from said subject is indicative that said subject does not have ovarian or rectal cancer or a predisposition to develop ovarian or rectal cancer.

In an embodiment, the control level is a level measured in a cancerous cell and/or tissue sample, and (i) a similar or higher level of expression in the ovarian or rectal cell and/or tissue sample from said subject is indicative that said subject has ovarian or rectal cancer or a predisposition to develop ovarian or rectal cancer; or (ii) a lower level of expression in the ovarian or rectal cell and/or tissue sample from said subject is indicative that said subject does not have ovarian or rectal cancer or a predisposition to develop ovarian or rectal cancer.

“Control level” or “reference level” or “standard level” are used interchangeably herein and broadly refers to a separate baseline level measured in a comparable “control” sample, which is generally from a subject not suffering from the disease (rectal or ovarian cancer) or not at risk of suffering from the disease. Alternatively, in another embodiment, the comparable “control” sample is from a subject not suffering the disease (rectal or ovarian cancer) or at risk of suffering from the disease. The corresponding control level may be a level corresponding to an average or median level calculated based of the levels measured in several reference or control subjects (e.g., a pre-determined or established standard level). The control level may be a pre-determined “cut-off” value recognized in the art or established based on levels measured in samples from one or a group of control subjects. The corresponding reference/control level may be adjusted or normalized for age, gender, race, or other parameters. The “control level” can thus be a single number/value, equally applicable to every patient individually, or the control level can vary, according to specific subpopulations of patients. Thus, for example, older men might have a different control level than younger men, and women might have a different control level than men. The predetermined standard level can be arranged, for example, where a tested population is divided equally (or unequally) into groups, such as a low-risk group, a medium-risk group and a high-risk group or into quadrants or quintiles, the lowest quadrant or quintile being individuals with the lowest risk (i.e., lowest amount of Galectin-7) and the highest quadrant or quintile being individuals with the highest risk (i.e., highest amount of Galectin-7).

It will also be understood that the control levels according to the invention may be, in addition to predetermined levels or standards, Galectin-7 levels measured in other samples (e.g. from healthy/normal subjects, or cancer patients) tested in parallel with the experimental sample.

In embodiments, the cut-off value may be determined using a Receiver Operator Curve, according to the method of Sackett et al., Clinical Epidemiology: A Basic Science for Clinical Medicine, pp. 106-7 (1985). Briefly, in this embodiment, the cut-off value may be determined from a plot of pairs of true positive rates (i.e., sensitivity) and false positive rates (100%-specificity) that correspond to each possible cut-off value for the diagnostic test result. The cut-off value on the plot that is the closest to the upper left-hand corner (i.e., the value that encloses the largest area) is the most accurate cut-off value, and a sample generating a signal that is higher or lower than the cut-off value determined by this method may be considered positive. Alternatively, the cut-off value may be shifted to the left along the plot, to minimize the false positive rate, or to the right, to minimize the false negative rate. In general, a sample generating a signal that is higher or lower than the cut-off value determined by this method is considered positive for a cancer.

As used herein, the term “predisposition” refers to the likelihood to develop the disorder or disease. An individual with a predisposition or susceptibility to a disorder or disease is more likely to develop the disorder or disease than an individual without the predisposition to the disorder or disease, or is more likely to develop the disorder or disease than members of a relevant general population under a given set of environmental conditions (diet, physical activity regime, geographic location, etc.).

“Higher expression” or “higher level of expression” as used herein refers to (i) higher expression of Galactin-7 (protein and/or mRNA) in one or more given cells present in the sample (relative to the control) and/or (ii) higher amount of Galactin-7-expressing/positive cells in the sample (relative to the control). “Lower expression” or “lower level of expression” as used herein refers to (i) lower expression of Galactin-7 (protein and/or mRNA) in one or more given cells present in the sample (relative to the control) and/or (ii) lower amount of Galactin-7-expressing/positive cells in the sample (relative to the control). In an embodiment, higher or lower refers to a level of expression that is at least one standard deviation above or below the control level (e.g., the predetermined cut-off value), and a “similar expression” or “similar level of expression” refers to a level of expression that is less than one standard deviation above or below the control level (e.g., the predetermined cut-off value). In embodiments, higher or lower refers to a level of expression that is at least 1.5, 2, 2.5 or 3 standard deviations above or below the control level (e.g., the predetermined cut-off value), and a “similar expression” or “similar level of expression” refers to a level of expression that is less than 1.5, 2, 2.5 or 3 standard deviation above or below the control level (e.g., the predetermined cut-off value). In other embodiments, a similar expression or similar level of expression. In another embodiment, “higher expression” refers to an expression that is at least 20, 25, 30, 35, 40, 45, or 50% higher in the test sample relative to the control level, or between the later (second) time point and the first time point. In an embodiment, “lower expression” refers to an expression that is at least 20, 25, 30, 35, 40, 45, or 50% lower in the test sample relative to the control level, or between the later (second) time point and the first time point. In an embodiment, “similar expression” refers to an expression that varies by less than 20, 15, or 10% between the test sample and the control level, or between the later (second) time point and the first time point.

Methods to measure the amount/level of proteins (Galectin-7) are well known in the art. Galectin-7 protein levels may be detected directly using a ligand binding specifically to human Galectin-7 protein (a Galectin-7 binding molecule or reagent), such as an antibody or a fragment thereof (for methods, see for example Harlow, E. and Lane, D (1988) Antibodies: A Laboratory Manual, Cold Spring Harbor Laboratory Press, Cold Spring Harbor, N.Y.). In embodiments, such a Galectin-7 binding molecule or reagent is labeled/conjugated, e.g., radio-labeled, chromophore-labeled, fluorophore-labeled, or enzyme-labeled to facilitate detection and quantification of the complex (direct detection). Alternatively, Galectin-7 protein levels may be detected indirectly, using a Galectin-7 binding molecule or reagent, followed by the detection of the [Galectin-7/Galectin-7 binding molecule or reagent] complex using a second ligand (or second binding molecule) specifically recognizing the Galectin-7 binding molecule or reagent (indirect detection). Such a second ligand may be radio-labeled, chromophore-labeled, fluorophore-labeled, or enzyme-labeled to facilitate detection and quantification of the complex. Enzymes used for labeling antibodies for immunoassays are known in the art, and the most widely used are horseradish peroxidase (HRP) and alkaline phosphatase (AP). Examples of Galectin-7 binding molecules or reagents include antibodies (monoclonal or polyclonal), natural or synthetic Galectin-7 ligands, glycoproteins, monosaccharides (e.g., galactose, galactosamine, lactose) aptamers and the like. The term “antibody” as used herein encompasses monoclonal antibodies, polyclonal antibodies, multispecific antibodies (e.g., bispecific antibodies), and antibody fragments, so long as they exhibit the desired biological activity or specificity (i.e. binding to Galectin-7). “Antibody fragments” comprise a portion of a full-length antibody, generally the antigen binding or variable region thereof. Anti-human Galectin-7 antibodies are well known in the art and are commercially available from several providers, for example Abcam™ (Cat. #ab89560), Epitomics™ (Cat. #: 2955-1), R&D Systems™ (Cat. #: MAB1339), BioVision™ (Cat. #: 5647-100), Santa Cruz Biotech™ (Cat. #: sc-166222), and Novus Biologicals™ (Cat. #: NBP1-19711). Antibody mimetics not based on immunoglobulin/antibody scaffolds may also be used as binding reagents, for example Affibody, DARPin, Anticalin, Avimer, Versabody, or Duocalin molecules.

Examples of methods to measure the amount/level of Galectin-7 protein in a sample include, but are not limited to: Western blot, immunoblot, enzyme-linked immunosorbent assay (ELISA), “sandwich” immunoassays, radioimmunoassay (RIA), immunoprecipitation, surface plasmon resonance (SPR), chemiluminescence, fluorescent polarization, phosphorescence, immunohistochemical (IHC) analysis, matrix-assisted laser desorption/ionization time-of-flight (MALDI-TOF) mass spectrometry, microcytometry, microarray, antibody array, microscopy (e.g., electron microscopy), flow cytometry, proteomic-based assays, and assays based on a property of Galectin-7 including but not limited to ligand binding or interaction with other protein partners. In an embodiment, the amount/level of Galectin-7 protein is measured by IHC analysis (e.g., on ovarian or rectal tissue sections). In a further embodiment, the IHC analysis is performed using an anti-Galectin-7 antibody, in a further embodiment a conjugated anti-Galectin-7 antibody.

In another embodiment, the present invention provides a method for diagnosing ovarian cancer or a predisposition to develop ovarian cancer in a subject, said method comprising: contacting an ovarian cell and/or tissue sample from said subject with a Galectin-7 binding reagent; measuring the amount/level of Galectin-7/Galectin-7 binding reagent complexes in the sample; diagnosing ovarian cancer or a predisposition to develop ovarian cancer based on the amount/level of Galectin-7/Galectin-7 binding reagent complexes in the sample.

In another embodiment, the present invention provides a method for diagnosing rectal cancer or a predisposition to develop rectal cancer in a subject, said method comprising: contacting a rectal cell and/or tissue sample from said subject with a Galectin-7 binding reagent (e.g., an anti-Galectin-7 antibody); measuring the amount/level of Galectin-7/Galectin-7 binding reagent complexes in the sample; diagnosing rectal cancer or a predisposition to develop rectal cancer based on the amount/level of Galectin-7/Galectin-7 binding reagent complexes in the sample.

In an embodiment, the amount/level of Galectin-7/Galectin-7 binding reagent complexes is measured by measuring (i) the intensity of the signal (e.g., staining intensity) and/or (ii) the proportion of Galectin-7-positive cells (e.g., the proportion of stained cells) in the sample.

In another embodiment, the method comprises determining the cellular localization or distribution (e.g., nuclear, mitochondrial, cytoplasmic) of Galectin-7 in the sample from the subject, and comparing the localization/distribution to a control (e.g., non-cancerous sample), wherein a difference in the cellular localization and/or distribution of Galectin-7 relative to a non-cancerous control is indicative that the subject has ovarian or rectal cancer or a predisposition to develop ovarian or rectal cancer.

In an embodiment, the level of expression of Galectin-7 is measured at the nucleic acid (mRNA or cDNA) level. In an embodiment, the above-mentioned method comprises contacting the subject's sample (containing nucleic acids) with one or more oligonucleotides (nucleic acid primer(s) or probe(s)) capable of hybridizing to a DNA or RNA that encodes Galectin-7 (SEQ ID NO:1), under conditions such that hybridization can occur, and detecting or measuring any resulting amplification and/or hybridization. In an embodiment, the oligonucleotide comprises at least 8 nucleotides, 12 nucleotides or 15 nucleotides. In an embodiment, the oligonucleotide has a length of 100, 75, 50, 40, 35, 30, 25 or 20 nucleotides or less. In an embodiment, the oligonucleotide comprises at least 8, 12 or 15 (consecutive) nucleotides from the sequence of SEQ ID NO: 1 (or from the complement thereof).

The levels of Galectin-7 nucleic acid can then be evaluated according to the methods disclosed below, e.g., with or without the use of nucleic acid amplification methods. In some embodiments, nucleic acid amplification methods can be used to detect Galectin-7. For example, the oligonucleotide primers and probes may be used in amplification and detection methods that use nucleic acid substrates isolated by any of a variety of well-known and established methodologies (e.g., Sambrook et al., Molecular Cloning, A laboratory Manual, pp. 7.37-7.57 (2nd ed., 1989); Lin et al., in Diagnostic Molecular Microbiology, Principles and Applications, pp. 605-16 (Persing et al., eds. (1993); Ausubel et al., Current Protocols in Molecular Biology (2001 and later updates thereto)). Methods for amplifying nucleic acids include, but are not limited to, for example the polymerase chain reaction (PCR) and reverse transcription PCR (RT-PCR) (see e.g., U.S. Pat. Nos. 4,683,195; 4,683,202; 4,800,159; 4,965,188), ligase chain reaction (LCR) (see, e.g., Weiss, Science 254: 1292-93 (1991)), strand displacement amplification (SDA) (see e.g., Walker et al, Proc. Natl. Acad. Sci. USA 89:392-396 (1992); U.S. Pat. Nos. 5,270,184 and 5,455,166), Thermophilic SDA (tSDA) (see e.g., European Pat. No. 0 684 315) and methods described in U.S. Pat. No. 5,130,238; Lizardi et al., BioTechnol. 6:1197-1202 (1988); Kwoh et al., Proc. Natl. Acad. Sci. USA 86:1173-77 (1989); Guatelli et al., Proc. Natl. Acad. Sci. USA 87:1874-78 (1990); U.S. Pat. Nos. 5,480,784; 5,399,491; U.S. Publication No. 2006/46265. The methods include the use of Transcription Mediated Amplification (TMA), which employs an RNA polymerase to produce multiple RNA transcripts of a target region (see, e.g., U.S. Pat. Nos. 5,480,784; 5,399,491 and US Publication No. 2006/46265).

“Nucleic acid hybridization” refers generally to the hybridization of two single-stranded nucleic acid molecules having complementary base sequences, which under appropriate conditions will form a thermodynamically favored double-stranded structure. Examples of hybridization conditions can be found in the two laboratory manuals referred above (Sambrook et al., 1989, supra and Ausubel, et al. (eds), 1989, Current Protocols in Molecular Biology, Vol. 1, Green Publishing Associates, Inc., and John Wiley & Sons, Inc., New York,) and are commonly known in the art. Hybridization to filter-bound sequences under moderately stringent conditions may, for example, be performed in 0.5 M NaHPO₄, 7% sodium dodecyl sulfate (SDS), 1 mM EDTA at 65° C., and washing in 0.2×SSC/0.1% SDS at 42° C. (see Ausubel, et al. (eds), 1989, Current Protocols in Molecular Biology, Vol. 1, Green Publishing Associates, Inc., and John Wiley & Sons, Inc., New York, at p. 2.10.3). Alternatively, hybridization to filter-bound sequences under stringent conditions may, for example, be performed in 0.5 M NaHPO₄, 7% SDS, 1 mM EDTA at 65° C., and washing in 0.1×SSC/0.1% SDS at 68° C. (see Ausubel, et al. (eds), 1989, supra). In other examples of hybridization, a nitrocellulose filter can be incubated overnight at 65° C. with a labeled probe specific to one or the other two alleles in a solution containing 50% formamide, high salt (5×SSC or 5× SSPE), 5× Denhardt's solution, 1% SDS, and 100 μg/ml denatured carrier DNA (i.e. salmon sperm DNA). The non-specifically binding probe can then be washed off the filter by several washes in 0.2×SSC/0.1% SDS at a temperature which is selected in view of the desired stringency: room temperature (low stringency), 42° C. (moderate stringency) or 65° C. (high stringency). Hybridization conditions may be modified in accordance with known methods depending on the sequence of interest (see Tijssen, 1993, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, New York). The selected temperature is based on the melting temperature (Tm) of the DNA hybrid (Sambrook et al. 1989, supra). Generally, stringent conditions are selected to be about 5° C. lower than the thermal melting point for the specific sequence at a defined ionic strength and pH.

The nucleic acid or amplification product may be detected or quantified by hybridizing a labeled probe to a portion of the Galectin-7 nucleic acid or amplified product. The labeled probe contains a detectable group that may be, for example, a fluorescent moiety, chemiluminescent moiety, radioisotope, biotin, avidin, enzyme, enzyme substrate, or other reactive group. Other well-known detection techniques include, for example, gel filtration, gel electrophoresis and visualization of the amplicons, and High Performance Liquid Chromatography (HPLC). In certain embodiments, for example using real-time TMA or real-time PCR, the level of amplified product is detected as the product accumulates. The detecting step may either be qualitative and/or quantitative, although in some embodiments quantitative detection of amplicons may be preferred, as the level of gene expression may be indicative of the aggressiveness, degree of metastasis, etc., of the ovarian or rectal cancer.

In another embodiment, the expression of Galectin-7 is indirectly measured by detecting the level of miRNAs that control the intracellular level of Galectin-7 mRNA.

In another embodiment, the expression of Galectin-7 is indirectly measured by measuring the level of methylation, or activation state, of the Galectin-7 promoter. The level of Galectin-7 promoter methylation has been shown to be correlated with Galectin-7 expression, i.e. hypomethylation of the promoter leads to Galectin-7 expression (Demers et al., BBRC, 2009). The level of methylation of DNA may be assessed using well known methods such as methylation-specific polymerase chain reaction (MS-PCR), bisulfite sequencing, Methylation-sensitive single-strand conformation analysis (MS-SSCA), and Methylation-sensitive single-nucleotide primer extension (MS-SnuPE), or using kits (e.g., EpiTect™ Methyl II PCR Primer Assay for Human LGALS7B (CpG Island 107444): EPHS107444-1A from SABiosciences)

In certain embodiments, the above-mentioned methods involve normalizing the level of expression of the Galectin-7 nucleic acid. Methods for normalizing the level of expression of a gene are well known in the art. For example, the expression level of Galectin-7 can be normalized on the basis of the relative ratio of the mRNA level of Galectin-7 to the mRNA level of a housekeeping gene or the relative ratio of the protein level of the Galectin-7 protein to the protein level of the housekeeping protein, so that variations In the sample extraction efficiency among cells or tissues are reduced in the evaluation of the gene expression level. A “housekeeping gene” is a gene the expression of which is substantially the same from sample to sample or from tissue to tissue, or one that is relatively refractory to change in response to external stimuli. A housekeeping gene can be any RNA molecule other than Galectin-7 RNA that will allow normalization of sample RNA or any other marker that can be used to normalize for the amount of total RNA added to each reaction. For example, the GAPDH gene, the G6PD gene, the actin gene, ribosomal RNA, 36B4 RNA, PGK1, RPLP0, or the like, may be used as a housekeeping gene.

In certain embodiments, the above-mentioned methods involve calibrating the level of expression of the Galectin-7 nucleic acid. Methods for calibrating the level of expression of a gene are well known in the art. For example, the expression of a gene can be calibrated using reference samples, which are commercially available. Examples of reference samples include, but are not limited to: Stratagene™ QPCR Human Reference Total RNA, Clontech™ Universal Reference Total RNA, and XpressRef™ Universal Reference Total RNA.

“Cell and/or tissue sample” refers to any solid or liquid sample isolated from a human and which contain cells from ovarian or rectal origin. In a particular embodiment, it refers to any solid or liquid sample isolated from a biopsy material (from an ovarian or rectal biopsy). The sample may be used directly or submitted to one or more treatments (washing, purification/enrichment steps, freezing/defreezing, paraffin embedding, etc.) prior to use. The sample may be fresh or frozen, paraffin embedded or deparaffinized. In an embodiment, the above-mentioned method further comprises: collecting a cell and/or tissue sample (an ovarian or rectal cell and/or tissue sample) from the subject, for example by performing an ovarian or rectal biopsy on the subject to obtain the cell and/or tissue sample to be analyzed for Galectin-7 expression.

In an embodiment, the above-mentioned method may be combined with other assays, methods and criteria for diagnosing ovarian or rectal cancer. In an embodiment, the above-noted method further comprises selecting a subject suspected of suffering from ovarian or rectal cancer, or suspected of being predisposed to developing ovarian or rectal cancer (e.g., based on family antecedents and/or other risk factors, for example).

In certain embodiments, methods of diagnosis described herein may be at least partly, or wholly, performed in vitro. In a further embodiment, the method is wholly performed in vitro.

In an embodiment, the above-mentioned method further comprises selecting and/or administering a course of therapy or prophylaxis to said subject in accordance with the diagnostic result. If it is determined that the subject has ovarian or rectal cancer or a predisposition to develop ovarian or rectal cancer, the method further comprises subjecting the subject to an anticancer therapy (e.g., surgery, radiation therapy and/or chemotherapy).

Accordingly, in another aspect, the present invention provides a method comprising: measuring the level of expression of Galectin-7 in an ovarian cell and/or tissue sample from said subject; comparing said level of expression to a control level; determining whether said subject has ovarian cancer or a predisposition to develop ovarian cancer based on said comparison; and if said subject has ovarian cancer or a predisposition to develop ovarian cancer, subjecting the subject to an anticancer therapy (e.g., surgery, radiation therapy and/or chemotherapy).

The invention also provides diagnostic kits, comprising a Galectin-7 binding reagent (e.g., an anti-Galectin-7 antibody). In addition, such a kit may optionally comprise one or more of the following: (1) instructions for using the Galectin-7 binding reagent for the diagnosis, prognosis, therapeutic monitoring of ovarian or rectal cancer, or any combination of these applications; (2) a labeled binding partner to the Galectin-7 binding reagent; (3) one or more reagents useful to perform the method (buffers, solutions, enzymes, etc.); (4) one or more containers; and/or (5) appropriate controls/standards. If no labeled binding partner to the Galectin-7 binding reagent is provided, the Galectin-7 binding reagent itself can be labeled with a detectable marker, e.g. a chemiluminescent, enzymatic, fluorescent, or radioactive moiety.

The invention also provides a kit comprising one or more oligonucleotides (e.g., a nucleic acid probe and/or a pair of primers) capable of hybridizing to and/or amplifying a nucleic acid encoding Galectin-7. In a specific embodiment, the kit may optionally comprise one or more of the following: (1) instructions for using the one or more oligonucleotides for the diagnosis, prognosis, therapeutic monitoring of ovarian or rectal cancer, or any combination of these applications; (2) one or more reagents useful to perform the method (buffers, solutions, enzymes, etc.); (3) one or more containers; and/or (4) appropriate controls/standards. For example, the kit may optionally further comprise a predetermined amount of a nucleic acid encoding Galectin-7, e.g. for use as a standard or control.

In some aspects, the present invention provides methods, assays, systems, and code for classifying whether a sample is associated with ovarian or rectal cancer using a statistical algorithm or process to classify the sample as an ovarian or rectal cancer sample or non-ovarian or rectal cancer sample.

In another aspect, the present invention provides an ovarian or rectal cancer diagnostic system comprising (i) an ovarian or rectal cell and/or tissue sample; (ii) a Galectin-7 binding reagent (in contact with the ovarian or rectal cell and/or tissue sample); and (iii) a device for detecting the presence and/or amount of Galectin-7/Galectin-7 binding reagent complexes (e.g., a spectrometer, a microscope, a flow cytometer). In an embodiment, the above-mentioned system further comprises an algorithm (e.g., a statistical algorithm) for analyzing the Galectin-7 expression data (profile), and classifying the sample from the subject as an ovarian or rectal cancer sample or non-ovarian or rectal cancer sample.

In some embodiments, methods for the diagnosis of ovarian or rectal cancer in a subject is based upon the diagnostic marker (Galectin-7) profile, alone or in combination with a symptom profile, in conjunction with a statistical algorithm. In certain instances, the statistical algorithm is a learning statistical classifier system. The learning statistical classifier system can be selected from the group consisting of a random forest (RF), classification and regression tree (C&RT), boosted tree, neural network (NN), support vector machine (SVM), general chi-squared automatic interaction detector model, interactive tree, multiadaptive regression spline, machine learning classifier, and combinations thereof. In certain embodiments, the methods comprise classifying a sample from the subject as an ovarian or rectal cancer sample or non-ovarian or rectal cancer sample.

As used herein, the term “profile” includes any set of data that represents the distinctive features or characteristics associated with ovarian or rectal cancer. The term encompasses a “Galectin-7 profile” that analyzes Galectin-7 expression/levels in a sample, a “symptom profile” that identifies one or more ovarian or rectal cancer-related clinical factors (i.e., symptoms) an individual is experiencing or has experienced, and combinations thereof. For example, a “Galectin-7 profile” can include a set of data that represents the presence or level of Galectin-7. Likewise, a “symptom profile” can include a set of data that represents the presence, severity, frequency, and/or duration of one or more symptoms associated with ovarian or rectal cancer.

In certain instances, the statistical algorithm is a single learning statistical classifier system. Preferably, the single learning statistical classifier system comprises a tree-based statistical algorithm such as a RF or C&RT. As a non-limiting example, a single learning statistical classifier system can be used to classify the sample as an ovarian or rectal cancer sample or non-ovarian or rectal cancer sample based upon a prediction or probability value and the presence or level of Galectin-7 (i.e., Galectin-7 profile), alone or in combination with the presence or severity of at least one symptom (i.e., symptom profile). The use of a single learning statistical classifier system typically classifies the sample as an ovarian or rectal cancer sample with a sensitivity, specificity, positive predictive value, negative predictive value, and/or overall accuracy of at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%. As such, the classification of a sample as an ovarian or rectal cancer sample or non-ovarian or rectal cancer sample is useful for aiding in the diagnosis of ovarian or rectal cancer in a subject.

In certain other instances, the statistical algorithm is a combination of at least two learning statistical classifier systems. Preferably, the combination of learning statistical classifier systems comprises a RF and a NN, e.g., used in tandem or parallel. As a non-limiting example, a RF can first be used to generate a prediction or probability value based upon the diagnostic marker (Galectin-7) profile, alone or in combination with a symptom profile, and a NN can then be used to classify the sample as an ovarian or rectal cancer sample or non-ovarian or rectal cancer sample based upon the prediction or probability value and the diagnostic marker (Galectin-7) profile. Advantageously, the hybrid RF/NN learning statistical classifier system of the present invention classifies the sample as an ovarian or rectal cancer sample with a sensitivity, specificity, positive predictive value, negative predictive value, and/or overall accuracy of at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

In some instances, the data obtained from using the learning statistical classifier system or systems can be processed using a processing algorithm. Such a processing algorithm can be selected, for example, from the group consisting of a multilayer perceptron, backpropagation network, and Levenberg-Marquardt algorithm. In other instances, a combination of such processing algorithms can be used, such as in a parallel or serial fashion.

In certain other embodiments, the methods of the present invention further comprise sending the ovarian or rectal cancer classification results to a clinician, e.g., an oncologist or a general practitioner. In another embodiment, the methods of the present invention provide a diagnosis in the form of a probability that the individual has ovarian or rectal cancer. For example, the individual can have about a 0%, 5%, 10%, 15%, 20%, 25%, 30%, 35%, 40%, 45%, 50%, 55%, 60%, 65%, 70%, 75%, 80%, 85%, 90%, 95%, or greater probability of having ovarian or rectal cancer. In yet another embodiment, the methods of the present invention further provide a prognosis of ovarian or rectal cancer in the individual. For example, the prognosis can be surgery, development of a category or clinical subtype of ovarian or rectal cancer, development of one or more symptoms, or recovery from the disease.

In one aspect, the present invention provides a computer-readable medium comprising code for controlling one or more processors to classify whether the cell or tissue sample from a subject is associated with ovarian or rectal cancer, the code comprising instructions to apply a statistical process to a data set comprising a diagnostic marker (Galectin-7) profile to produce a statistically derived decision classifying the sample as an ovarian or rectal cancer sample or non-ovarian or rectal cancer sample based upon the Galectin-7 profile, wherein the Galectin-7 profile indicates the level of Galectin-7.

In other embodiments, the computer-readable medium for ruling in ovarian or rectal cancer comprises instructions to apply a statistical process to a data set comprising a Galectin-7 profile optionally in combination with a symptom profile which indicates the presence or severity of at least one symptom in the individual to produce a statistically derived decision classifying the sample as an ovarian or rectal cancer sample or non-ovarian or rectal cancer sample based upon the Galectin-7 profile and the symptom profile. One skilled in the art will appreciate that the statistical process can be applied to the Galectin-7 profile and the symptom profile simultaneously or sequentially in any order.

In an embodiment, the present invention provides a computer-readable medium including code for controlling one or more processors to classify whether a cell or tissue sample from an individual is associated with ovarian or rectal cancer, the code comprising instructions to apply a statistical process to a data set comprising a Galectin-7 profile to produce a statistically derived decision classifying the sample as an ovarian or rectal cancer sample or non-ovarian or rectal cancer sample based upon the Galectin-7 profile, wherein the Galectin-7 profile indicates the presence or level of Galectin-7 in the sample.

In one embodiment, the computer-readable medium for ruling in ovarian or rectal cancer comprises instructions to apply a statistical process to a data set comprising a Galectin-7 profile optionally in combination with a symptom profile which indicates the presence or severity of at least one symptom in the individual to produce a statistically derived decision classifying the sample as an ovarian or rectal cancer sample or non-ovarian or rectal cancer sample based upon the Galectin-7 profile and the symptom profile.

In another aspect, the present invention provides a system for classifying whether a cell or tissue sample from a subject is associated with ovarian or rectal cancer, the system comprising: (a) a data acquisition module configured to produce a data set comprising a Galectin-7 profile, wherein the Galectin-7 profile indicates the presence or level of Galectin-7; (b) a data processing module configured to process the data set by applying a statistical process to the data set to produce a statistically derived decision classifying the sample as an ovarian or rectal cancer sample or non-ovarian or rectal cancer sample based upon the Galectin-7 profile; and (c) a display module configured to display the statistically derived decision.

In certain embodiments, the system for classifying whether a cell or tissue sample is associated with ovarian or rectal cancer, aiding in the diagnosis of ovarian or rectal cancer, or ruling in ovarian or rectal cancer comprises a data acquisition module configured to produce a data set comprising a Galectin-7 profile optionally in combination with a symptom profile which indicates the presence or severity of at least one symptom in the individual; a data processing module configured to process the data set by applying a statistical process to the data set to produce a statistically derived decision classifying the sample as an ovarian or rectal cancer sample or non-ovarian or rectal cancer sample based upon the Galectin-7 profile and the symptom profile; and a display module configured to display the statistically derived decision.

The term “statistical algorithm” or “statistical process” includes any of a variety of statistical analyses used to determine relationships between variables. In the present invention, the variables are the presence or level of Galectin-7 (optionally in combination with one or more additional marker), and, optionally, the presence or severity of at least one ovarian or rectal cancer-related symptom. Any number of markers and/or symptoms can be analyzed using a statistical algorithm described herein. For example, 1, 2, 3, 4, 5, 6, 7, 8, 9, 10, 11, 12, 13, 14, 15, 16, 17, 18, 19, 20, 25, 30, 35, 40, 45, 50, 55, 60, or more biomarkers and/or symptoms can be included in a statistical algorithm. In one embodiment, logistic regression is used. In another embodiment, linear regression is used. In certain instances, the statistical algorithms of the present invention can use a quantile measurement of a particular marker within a given population as a variable. Quantiles are a set of “cut points” that divide a sample of data into groups containing (as far as possible) equal numbers of observations. For example, quartiles are values that divide a sample of data into four groups containing (as far as possible) equal numbers of observations. The lower quartile is the data value a quarter way up through the ordered data set; the upper quartile is the data value a quarter way down through the ordered data set. Quintiles are values that divide a sample of data into five groups containing (as far as possible) equal numbers of observations. The present invention can also include the use of percentile ranges of marker levels (e.g., tertiles, quartile, quintiles, etc.), or their cumulative indices (e.g., quartile sums of marker levels, etc.) as variables in the algorithms (just as with continuous variables).

In an embodiment, the statistical algorithms of the present invention comprise one or more learning statistical classifier systems. As used herein, the term “learning statistical classifier system” includes a machine learning algorithmic technique capable of adapting to complex data sets (e.g., panel of markers of interest and/or list of symptoms) and making decisions based upon such data sets. In some embodiments, a single learning statistical classifier system such as a classification tree (e.g., random forest) is used. In other embodiments, a combination of 2, 3, 4, 5, 6, 7, 8, 9, 10, or more learning statistical classifier systems are used, preferably in tandem. Examples of learning statistical classifier systems include, but are not limited to, those using inductive learning (e.g., decision/classification trees such as random forests, classification and regression trees (C&RT), boosted trees, etc.), Probably Approximately Correct (PAC) learning, connectionist learning (e.g., neural networks (NN), artificial neural networks (ANN), neuro fuzzy networks (NFN), network structures, perceptrons such as multi-layer perceptrons, multi-layer feed-forward networks, applications of neural networks, Bayesian learning in belief networks, etc.), reinforcement learning (e.g., passive learning in a known environment such as naive learning, adaptive dynamic learning, and temporal difference learning, passive learning in an unknown environment, active learning in an unknown environment, learning action-value functions, applications of reinforcement learning, etc.), and genetic algorithms and evolutionary programming. Other learning statistical classifier systems include support vector machines (e.g., Kernel methods), multivariate adaptive regression splines (MARS), Levenberg-Marquardt algorithms, Gauss-Newton algorithms, mixtures of Gaussians, gradient descent algorithms, and learning vector quantization (LVQ).

Random forests are learning statistical classifier systems that are constructed using an algorithm developed by Leo Breiman and Adele Cutler. Random forests use a large number of individual decision trees and decide the class by choosing the mode (i.e., most frequently occurring) of the classes as determined by the individual trees. Random forest analysis can be performed, e.g., using the RandomForests™ software available from Salford Systems (San Diego, Calif.). See, e.g., Breiman, Machine Learning, 45:5-32 (2001); and http://stat-www.berkeley.edu/users/breiman/RandomForests/cc_home.htm, for a description of random forests.

Classification and regression trees represent a computer intensive alternative to fitting classical regression models and are typically used to determine the best possible model for a categorical or continuous response of interest based upon one or more predictors. Classification and regression tree analysis can be performed, e.g., using the CART software available from Salford Systems or the Statistica data analysis software available from StatSoft, Inc. (Tulsa, Okla.). A description of classification and regression trees is found, e.g., in Breiman et al. “Classification and Regression Trees,” Chapman and Hall, New York (1984); and Steinberg et al., “CART: Tree-Structured Non-Parametric Data Analysis,” Salford Systems, San Diego, (1995).

Neural networks are interconnected groups of artificial neurons that use a mathematical or computational model for information processing based on a connectionist approach to computation. Typically, neural networks are adaptive systems that change their structure based on external or internal information that flows through the network. Specific examples of neural networks include feed-forward neural networks such as perceptrons, single-layer perceptrons, multi-layer perceptrons, backpropagation networks, ADALINE networks, MADALINE networks, Learnmatrix networks, radial basis function (RBF) networks, and self-organizing maps or Kohonen self-organizing networks; recurrent neural networks such as simple recurrent networks and Hopfield networks; stochastic neural networks such as Boltzmann machines; modular neural networks such as committee of machines and associative neural networks; and other types of networks such as instantaneously trained neural networks, spiking neural networks, dynamic neural networks, and cascading neural networks. Neural network analysis can be performed, e.g., using the Statistica data analysis software available from StatSoft, Inc. See, e.g., Freeman et al., In “Neural Networks: Algorithms, Applications and Programming Techniques,” Addison-Wesley Publishing Company (1991); Zadeh, Information and Control, 8:338-353 (1965); Zadeh, “IEEE Trans. on Systems, Man and Cybernetics,” 3:28-44 (1973); Gersho et al., In “Vector Quantization and Signal Compression,” Kluywer Academic Publishers, Boston, Dordrecht, London (1992); and Hassoun, “Fundamentals of Artificial Neural Networks,” MIT Press, Cambridge, Mass., London (1995), for a description of neural networks.

Support vector machines are a set of related supervised learning techniques used for classification and regression and are described, e.g., in Cristianini et al., “An Introduction to Support Vector Machines and Other Kernel-Based Learning Methods,” Cambridge University Press (2000). Support vector machine analysis can be performed, e.g., using the SVM.sup.light software developed by Thorsten Joachims (Cornell University) or using the LIBSVM software developed by Chih-Chung Chang and Chih-Jen Lin (National Taiwan University).

The learning statistical classifier systems described herein can be trained and tested using a cohort of samples (e.g., cell or tissue samples) from healthy individuals, ovarian or rectal cancer patients. For example, samples from patients diagnosed by a physician, and preferably by an oncologist as having cell or tissue samples using a biopsy, for example, are suitable for use in training and testing the learning statistical classifier systems of the present invention. Samples from healthy individuals can include those that were not identified as ovarian or rectal cancer samples. One skilled in the art will know of additional techniques and diagnostic criteria for obtaining a cohort of patient samples that can be used in training and testing the learning statistical classifier systems of the present invention.

As used herein, the term “sensitivity” refers to the probability that a diagnostic method, system, or code of the present invention gives a positive result when the sample is positive, e.g., having ovarian or rectal cancer. Sensitivity is calculated as the number of true positive results divided by the sum of the true positives and false negatives. Sensitivity essentially is a measure of how well a method, system, or code of the present invention correctly identifies those with ovarian or rectal cancer from those without the disease. The statistical algorithms can be selected such that the sensitivity of classifying ovarian or rectal cancer is at least about 60%, and can be, for example, at least about 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

The term “specificity” refers to the probability that a diagnostic method, system, or code of the present invention gives a negative result when the sample is not positive, e.g., not having ovarian or rectal cancer. Specificity is calculated as the number of true negative results divided by the sum of the true negatives and false positives. Specificity essentially is a measure of how well a method, system, or code of the present invention excludes those who do not have ovarian or rectal cancer from those who have the disease. The statistical algorithms can be selected such that the specificity of classifying ovarian or rectal cancer is at least about 70%, for example, at least about 75%, 80%, 85%, 86%, 87%, 88%, 89%, 90%, 91%, 92%, 93%, 94%, 95%, 96%, 97%, 98%, or 99%.

As used herein, the term “negative predictive value” or “NPV” refers to the probability that an individual identified as not having ovarian or rectal cancer actually does not have the disease. Negative predictive value can be calculated as the number of true negatives divided by the sum of the true negatives and false negatives. Negative predictive value is determined by the characteristics of the diagnostic method, system, or code as well as the prevalence of the disease in the population analyzed. The statistical algorithms can be selected such that the negative predictive value in a population having a disease prevalence is in the range of about 70% to about 99% and can be, for example, at least about 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

The term “positive predictive value” or “PPV” refers to the probability that an individual identified as having ovarian or rectal cancer actually has the disease. Positive predictive value can be calculated as the number of true positives divided by the sum of the true positives and false positives. Positive predictive value is determined by the characteristics of the diagnostic method, system, or code as well as the prevalence of the disease in the population analyzed. The statistical algorithms can be selected such that the positive predictive value in a population having a disease prevalence is in the range of about 80% to about 99% and can be, for example, at least about 80%, 85%, 86%, 87%, 88%, 89%, 90%, 91%, 92%, 93%, 94%, 95%, 96%, 97%, 98%, or 99%.

Predictive values, including negative and positive predictive values, are influenced by the prevalence of the disease in the population analyzed. In the methods, systems, and code of the present invention, the statistical algorithms can be selected to produce a desired clinical parameter for a clinical population with a particular ovarian or rectal cancer prevalence. For example, learning statistical classifier systems can be selected for an ovarian or rectal cancer prevalence of up to about 1%, 2%, 3%, 4%, 5%, 6%, 7%, 8%, 9%, 10%, 15%, 20%, 25%, 30%, 35%, 40%, 45%, 50%, 55%, 60%, 65%, or 70%, which can be seen, e.g., in a clinician's office such as an oncologist's office or a general practitioner's office.

As used herein, the term “overall agreement” or “overall accuracy” refers to the accuracy with which a method, system, or code of the present invention classifies a disease state. Overall accuracy is calculated as the sum of the true positives and true negatives divided by the total number of sample results and is affected by the prevalence of the disease in the population analyzed. For example, the statistical algorithms can be selected such that the overall accuracy in a patient population having a disease prevalence is at least about 60%, and can be, for example, at least about 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

In an embodiment, the present invention relates to a Disease Classification System (DCS) for ovarian or rectal cancer. Examples of a DCS are described, for example, in US Patent publications Nos. 2008/0085524 (more particularly FIG. 2) and 2012/0315630 (more particularly FIG. 13). Such DCS includes a DCS intelligence module, such as a computer, having a processor and memory module. The intelligence module also includes communication modules for transmitting and receiving information over one or more direct connections (e.g., USB, Firewire, or other interface) and one or more network connections (e.g., including a modem or other network interface device). The memory module may include internal memory devices and one or more external memory devices. The intelligence module also includes a display module, such as a monitor or printer. In one aspect, the intelligence module receives data such as patient test results from a data acquisition module such as a test system, either through a direct connection or over a network. For example, the test system may be configured to run multianalyte tests on one or more patient samples and automatically provide the test results to the intelligence module. The data may also be provided to the intelligence module via direct input by a user or it may be downloaded from a portable medium such as a compact disk (CD), a USB storage device (e.g., USB flash drive) or a digital versatile disk (DVD). The test system may be integrated with the intelligence module, directly coupled to the intelligence module, or it may be remotely coupled with the intelligence module over the network. The intelligence module may also communicate data to and from one or more client systems over the network as is well known. For example, a requesting physician or healthcare provider may obtain and view a report from the intelligence module, which may be resident in a laboratory or hospital, using a client system.

The network can be a LAN (local area network), WAN (wide area network), wireless network, point-to-point network, star network, token ring network, hub network, or other configuration. As the most common type of network in current use is a TCP/IP (Transfer Control Protocol and Internet Protocol) network such as the global internetwork of networks often referred to as the “Internet”, but it should be understood that the networks that the present invention might use are not so limited, although TCP/IP is the currently preferred protocol.

Several elements in the system shown in FIG. 2 from US Patent Publication No. 2008/0085524 may include conventional, well-known elements that need not be explained in detail here. For example, the intelligence module could be implemented as a desktop personal computer, workstation, mainframe, laptop, etc. Each client system could include a desktop personal computer, workstation, laptop, PDA, cell phone, or any WAP-enabled device or any other computing device capable of interfacing directly or indirectly to the Internet or other network connection. A client system typically runs an HTTP client, e.g., a browsing program, such as Microsoft's Internet Explorer™ browser, Mozilla Firefox™, Opera's browser, or a WAP-enabled browser in the case of a cell phone, PDA or other wireless device, or the like, allowing a user of the client system to access, process, and view information and pages available to it from the intelligence module over the network. Each client system also typically includes one or more user interface devices, such as a keyboard, a mouse, touch screen, pen or the like, for interacting with a graphical user interface (GUI) provided by the browser on a display (e.g., monitor screen, LCD display, etc.) in conjunction with pages, forms, and other information provided by the intelligence module. As discussed above, the present invention is suitable for use with the Internet. However, it should be understood that other networks can be used instead of the Internet, such as an intranet, an extranet, a virtual private network (VPN), a non-TCP/IP based network, any LAN or WAN, or the like.

According to an embodiment, each client system and all of its components are operator configurable using applications, such as a browser, including computer code run using a central processing unit such as an Intel Pentium™ processor or the like. Similarly, the intelligence module and all of its components might be operator configurable using application(s) including computer code run using a central processing unit such as an Intel Pentium™ processor or the like, or multiple processor units. Computer code for operating and configuring the intelligence module to process data and test results as described herein is preferably downloaded and stored on a hard disk, but the entire program code, or portions thereof, may also be stored in any other volatile or non-volatile memory medium or device as is well known, such as a ROM or RAM, or provided on any other computer readable medium capable of storing program code, such as a compact disk (CD) medium, digital versatile disk (DVD) medium, a floppy disk, a USB storage device (e.g., USB flash drive), ROM, RAM, and the like.

The computer code for implementing various aspects and embodiments of the present invention can be implemented in any programming language that can be executed on a computer system such as, for example, in C, C⁺⁺, HTML, Java™, JavaScript™, or any other scripting language, such as VBScript. Additionally, the entire program code, or portions thereof, may be embodied as a carrier signal, which may be transmitted and downloaded from a software source (e.g., server) over the Internet, or over any other conventional network connection as is well known (e.g., extranet, VPN, LAN, etc.) using any communication medium and protocols (e.g., TCP/IP, HTTP, HTTPS, Ethernet, etc.) as are well known.

According to an embodiment, the intelligence module implements a disease classification process for analyzing patient test results and/or questionnaire responses to determine whether a patient sample is associated with ovarian or rectal cancer. The data may be stored in one or more data tables or other logical data structures in memory or in a separate storage or database system coupled with the intelligence module. One or more statistical processes are typically applied to a data set including test data for a particular patient. For example, the test data might include a Galectin-7 profile, which comprises data indicating the presence or level of Galectin-7 in a sample from the patient. The test data might also include a symptom profile, which comprises data indicating the presence or severity of at least one symptom associated with ovarian or rectal cancer that the patient is experiencing or has recently experienced. In one aspect, a statistical process produces a statistically derived decision classifying the patient sample as an ovarian or rectal cancer sample or non-ovarian or rectal cancer sample based upon the Galectin-7 profile and/or symptom profile. The statistically derived decision may be displayed on a display device associated with or coupled to the intelligence module, or the decision(s) may be provided to and displayed at a separate system, e.g., a client system. The displayed results allow a physician to make a reasoned diagnosis or prognosis.

In an embodiment, the above-mentioned ovarian or rectal cancer is an aggressive type of ovarian or rectal cancer, for example a malignant or metastatic type. In an embodiment, the ovarian cancer is a mucinous carcinoma, a transitional cell carcinoma or an adenocarcinoma (e.g., endometrioid adenocarcinoma).

In certain embodiments the above-mentioned data set further comprises a profile for one or more additional diagnostic markers associated ovarian or rectal cancer.

As used herein the term “subject” is meant to refer to any animal, such as a mammal including human, mice, rat, dog, cat, pig, cow, monkey, horse, etc. In an embodiment, the above-mentioned subject is a human.

### DESCRIPTION OF ILLUSTRATIVE EMBODIMENTS

The present invention is illustrated in further details by the following non-limiting examples.

### EXAMPLE 1

### Materials and Methods

To measure Galectin-7 expression in biopsies of patients by immunohistochemistry, tissue sections were blocked in 1% bovine serum albumin and 5% N-hydroxysuccinimide in 1× PBS and incubated overnight at 4° C. with a goat anti-human Galectin-7 polyclonal antibody (goat anti-human galectin-7 antibody from R & D Systems, Cat. No AF1339). Stainings were performed by using the Discovery™ XT automated immunostainer (Ventana Medical Systems, Tucson, Ariz.) on deparaffinized sections incubated in EDTA buffer (pH 8) for antigen retrieval. To reveal the reaction, DABmap™ (brown) or REDmap™ (red) kits were used (Ventana Medical Systems), and the slides were counterstained with hematoxylin. Each section was then scanned at high resolution (Nanozoomer™, Hammamatsu Photonics K.K.). Tissue sections were scored using an Allred scoring system accounting for both the intensity of staining (0=none, 1=weak, 2=moderate, 3=strong) and the proportion of stained cells (0=0%, 1=<1%, 2=1 to 10%, 3=11 to 33%, 4=34 to 66%, 5=>66%) producing a sum score of the two values (intensity+proportion=0 to 8).

### EXAMPLE 2

### Results

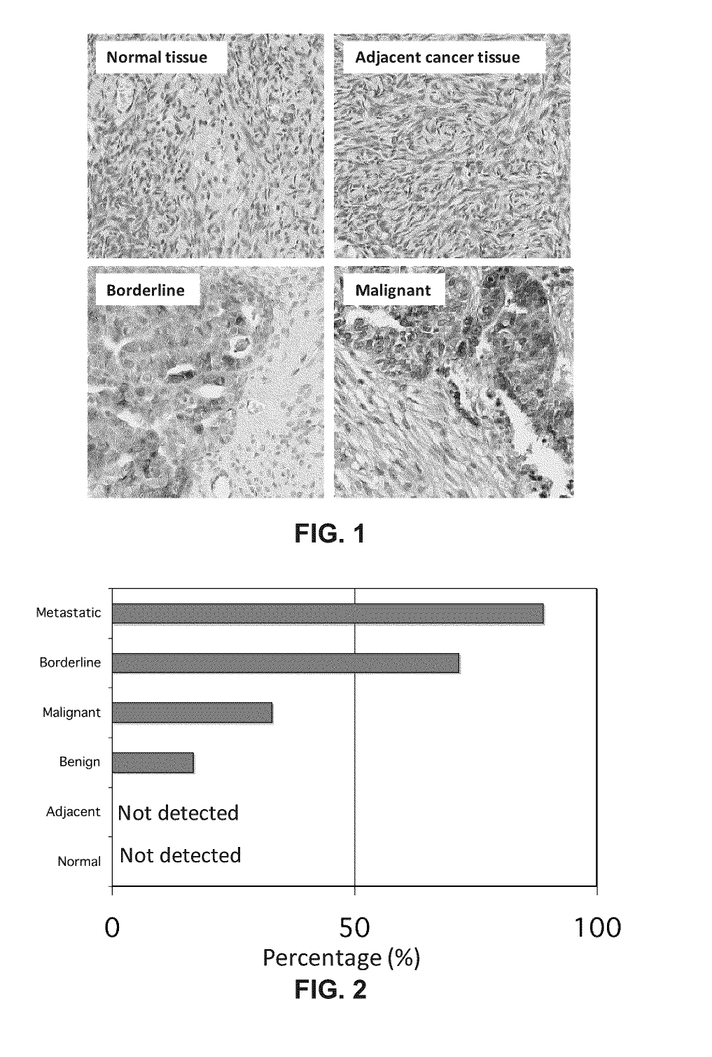

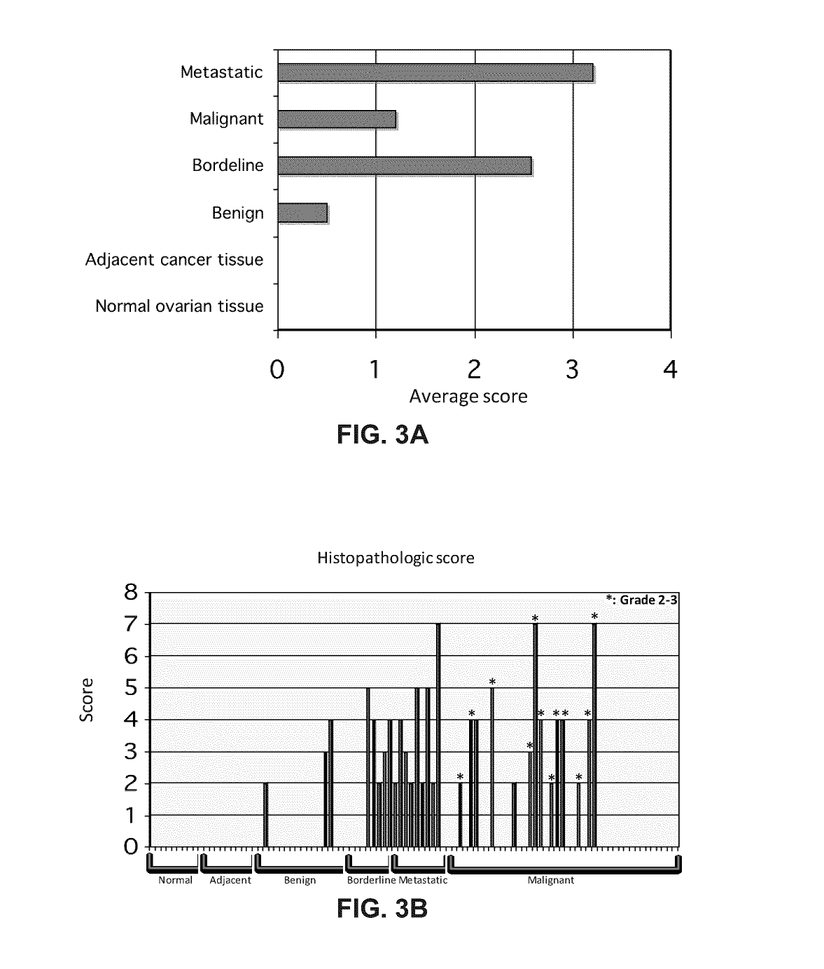

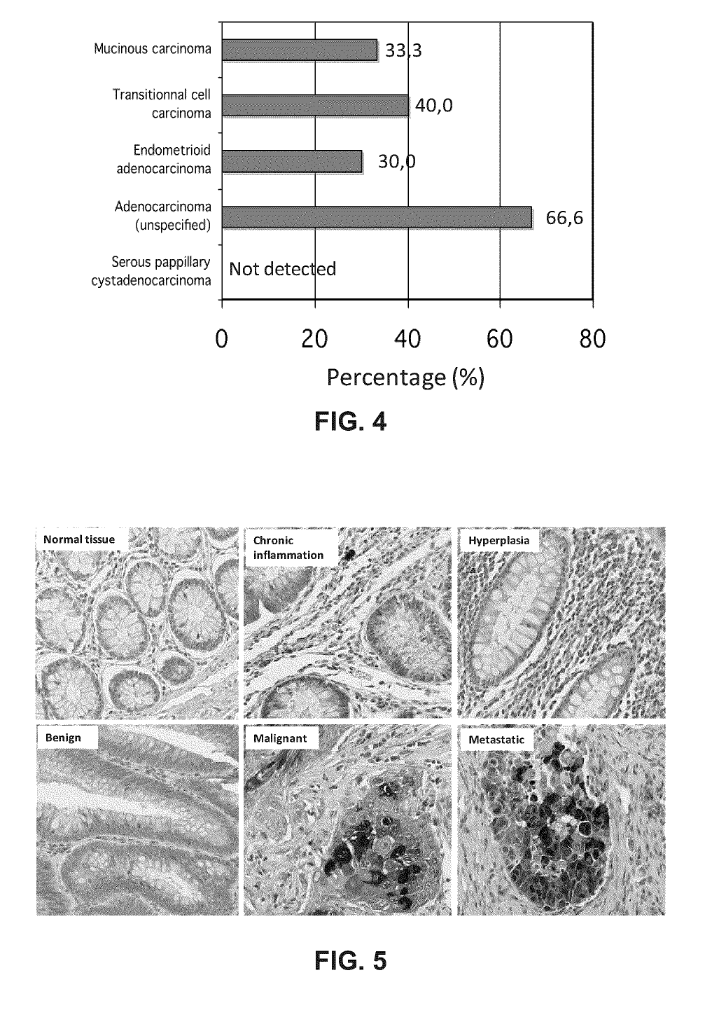

FIG. 1 shows an immunohistological analysis of human normal ovary tissues (upper left panel), cancer adjacent tissues (upper right panel), borderline (lower left panel) and malignant ovary tissues (lower right panel) showing absence of expression of Galectin-7 (upper left panel). The presence of cytoplasmic and nuclear Galectin-7 protein immunoreactivity was detected in borderline and malignant ovary tissues Galectin-7, but not in normal ovary tissues or cancer adjacent tissues. Also, galectin-7 expression appears to correlate with the aggressiveness of the cancer, with stronger expression in aggressive types of ovarian cancer, notably metastatic ovarian cancer (FIG. 2). Scoring of tissue microarrays constructed from human ovarian tissue specimens were stained with an anti-Galectin-7 antibody. Specimens were scored using an Allred scoring system (Allred et al., Mod Pathol. 1998; 11:155-168) accounting for both the intensity of staining and the proportion of stained cells producing a sum score of the two values (intensity+proportion=0 to 8). Again, aggressive types of ovarian cancer tend to exhibit a higher score relative to normal or benign tissues (FIGS. 3A and 3B). FIG. 4 shows that Galectin-7 is expressed in specific types of malignant ovarian cancers, namely mucinous carcinomas, transitional cell carcinomas, endometrioid adenocarcinomas and other unspecified adenocarcinomas, but not in serous papillary cystadenocarcinomas.

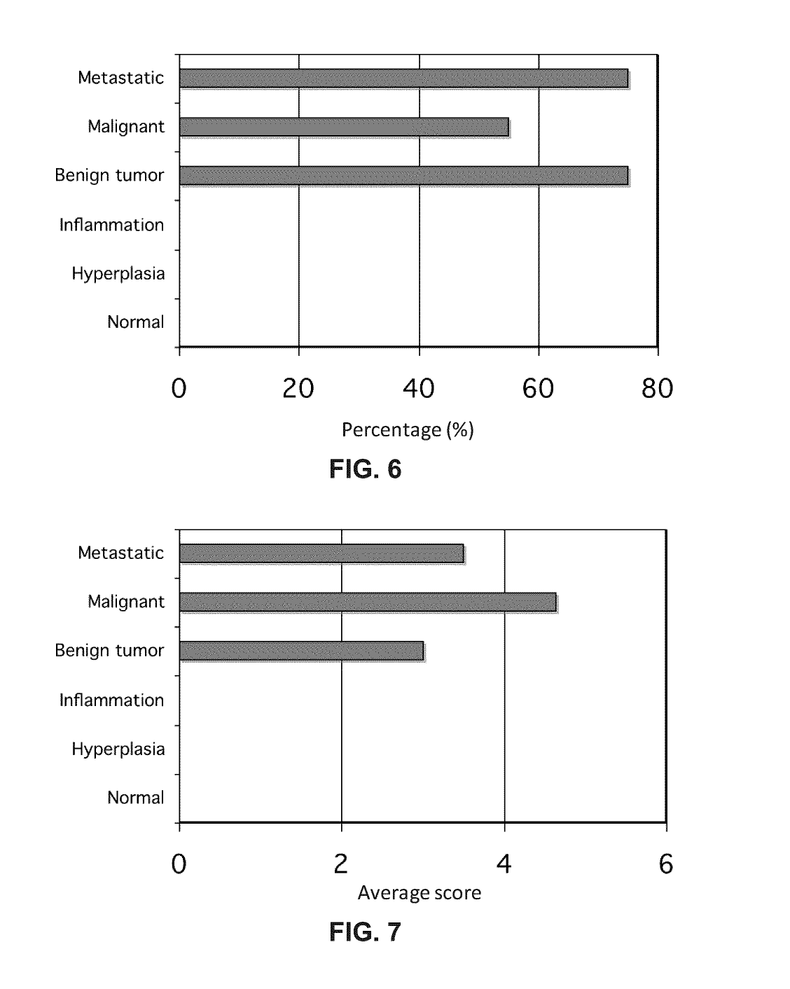

FIG. 5 shows an immunohistological analysis of human normal rectal tissues (upper left panel), chronic inflammation (upper middle panel), hyperplasia (upper right panel), benign rectal cancer (lower left panel), malignant cancer tissues (lower middle panel) and metastasic (lower right panel) rectal cancer tissues. No Galectin-7 expression was detected in human normal rectal tissues, and background staining is detected in interstitial cells in rectal tissue with chronic inflammation and hyperplasia. No positive staining was detected in ductal epithelial cells. Low expression of galectin-7 was found in epithelial cells of benign rectal cancer. However, strong cytoplasmic and nuclear Galectin-7 protein immunoreactivity was detected in malignant and metastatic rectal cancer tissues. FIGS. 6 and 7 show that Galectin-7 is preferentially expressed in abnormal rectal tissues (benign, malignant and metastatic tumors.

The scope of the claims should not be limited by the preferred embodiments set forth in the examples, but should be given the broadest interpretation consistent with the description as a whole.

### REFERENCES

1. American Cancer Society: Cancer Facts and Figures 2012. Atlanta, Ga.: American Cancer Society, 2012
2. Barondes S H, Cooper D N, Gitt M A, Leffler H. 1994. Galectins. Structure and function of a large family of animal lectins. J Biol Chem 269:20807.
3. Kasai K, Hirabayashi J. 1996. Galectins: a family of animal lectins that decipher glycocodes. J Biochem 119:1.
4. Kopitz J, André S, von Reitzenstein C, Versluis K, Kaltner H, Pieters R J, Wasano K, Kuwabara I, Liu F T, Cantz M, Heck A J, Gabius H J. Homodimeric galectin-7 (p53-induced gene 1) is a negative growth regulator for human neuroblastoma cells. Oncogene. 2003; 22:6277.
5. Kuwabara I, Kuwabara Y, Yang R Y, Schuler M, Green D R, Zuraw B L, Hsu D K, Liu F T. Galectin-7 (PIG1) exhibits pro-apoptotic function through JNK activation and mitochondrial cytochrome c release. J Biol Chem. 2002; 277:3487.
6. Liu F T, Rabinovich G A. Galectins as modulators of tumor progression. Nat Rev Cancer. 2005; 5:29.
7. Madsen P, Rasmussen H H, Flint T, Gromov P, Kruse T A, Honoré B, Vorum H, Celis J E. Cloning, expression, and chromosome mapping of human galectin-7. J Biol Chem. 1995; 270:5823
8. Ueda S, Kuwabara I, Liu F T. Suppression of tumor growth by galectin-7 gene transfer. Cancer Res. 2004; 64:5672.
9. Demers M, Couillard J, Giglia-Mari G, Magnaldo T, St-Pierre Y. Increased galectin-7 gene expression in lymphoma cells is under the control of DNA methylation. Biochem Biophys Res Commun. 2009 Sep. 25; 387(3):425-9. Epub 2009 Jul. 9.

1. A method for determining whether a subject has ovarian cancer or a predisposition to develop ovarian cancer, said method comprising:
(i) measuring the level of expression of Galectin-7 in an ovarian cell and/or tissue sample from said subject;
(ii) comparing said level of expression to a control level; and
(iii) determining whether said subject has ovarian cancer or a predisposition to develop ovarian cancer based on said comparison.

2. The method of claim 1, wherein (a) said control level is a level measured in a non-cancerous ovarian cell and/or tissue sample, and (i) a higher level of expression in the ovarian cell and/or tissue sample from said subject is indicative that said subject has ovarian cancer or a predisposition to develop ovarian cancer; or (ii) a similar or lower level of expression in the ovarian cell and/or tissue sample from said subject is indicative that said subject does not have ovarian cancer or a predisposition to develop ovarian cancer; or (b) said control level is a level measured in a cancerous ovarian cell and/or tissue sample, and (i) a similar or higher level of expression in the ovarian cell and/or tissue sample from said subject is indicative that said subject has ovarian cancer or a predisposition to develop ovarian cancer; or (ii) a lower level of expression in the ovarian cell and/or tissue sample from said subject is indicative that said subject does not have ovarian cancer or a predisposition to develop ovarian cancer.

3. (canceled)

4. The method of claim 1, wherein the level of expression of Galectin-7 is measured at the protein level.

5. (canceled)

6. The method of claim 4, wherein the level of expression of Galectin-7 is measured using an antibody.

7. The method of claim 4, wherein the level of expression of Galectin-7 is measured by immunohistochemistry.

8. The method of claim 1, wherein said ovarian cell and/or tissue sample is a biopsy sample.

9. The method of claim 1, wherein said ovarian cancer is a mucinous carcinoma, a transitional cell carcinoma or an adenocarcinoma.

10. The method of claim 9, wherein said adenocarcinoma is endometrioid adenocarcinoma.

11. A method for determining whether a subject has rectal cancer or a predisposition to develop rectal cancer, said method comprising:
(iv) measuring the level of expression of Galectin-7 in a rectal cell and/or tissue sample from said subject;
(v) comparing said level of expression to a control level; and
(vi) determining whether said subject has rectal cancer or a predisposition to develop rectal cancer based on said comparison.

12. The method of claim 11, wherein (a) said control level is a level measured in a non-cancerous rectal cell and/or tissue sample, and (i) a higher level of expression in the rectal cell and/or tissue sample from said subject is indicative that said subject has rectal cancer or a predisposition to develop rectal cancer; or (ii) a similar or lower level of expression in the rectal cell and/or tissue sample from said subject is indicative that said subject does not have rectal cancer or a predisposition to develop rectal cancer; or (b) said control level is a level measured in a cancerous rectal cell and/or tissue sample, and (i) a similar or higher level of expression in the rectal cell and/or tissue sample from said subject is indicative that said subject has rectal cancer or a predisposition to develop rectal cancer; or (ii) a lower level of expression in the rectal cell and/or tissue sample from said subject is indicative that said subject does not have rectal cancer or a predisposition to develop rectal cancer.

13. (canceled)

14. The method of claim 11, wherein the level of expression of Galectin-7 is measured at the protein level.

15. (canceled)

16. The method of claim 14, wherein the level of expression of Galectin-7 is measured using an antibody.

17. The method of claim 14, wherein the level of expression of Galectin-7 is measured by immunohistochemistry.

18. The method of claim 11, wherein said rectal cell and/or tissue sample is a biopsy sample.

19. A method for monitoring the progression of ovarian or rectal cancer in a subject, the method comprising:
(i) measuring the level of expression of Galectin-7 in a first ovarian or rectal cell and/or tissue sample from said subject at a first time point;
(ii) measuring the level of expression of Galectin-7 in a second ovarian or rectal cell and/or tissue sample from said subject at a later time point;
(iii) wherein (a) a level of expression of Galectin-7 that is higher in said second sample relative to said first sample is indicative that said ovarian or rectal cancer has progressed; (b) a level of expression of Galectin-7 that is lower in said second sample relative to said first sample is indicative that said ovarian or rectal cancer has regressed; or (c) a level of expression of Galectin-7 that is similar in said second sample relative to said first sample is indicative that said ovarian or rectal cancer is stable.

20. The method of claim 19, wherein the level of expression of Galectin-7 is measured at the protein level.

21. (canceled)

22. The method of claim 20, wherein the level of expression of Galectin-7 is measured using an antibody.

23. The method of claim 20, wherein the level of expression of Galectin-7 is measured by immunohistochemistry.

24-30. (canceled)

31. An ovarian or rectal cancer diagnostic system comprising (i) an ovarian or rectal cell and/or tissue sample; (ii) a Galectin-7 binding reagent; and (iii) a device for detecting the presence and/or amount of Galectin-7/Galectin-7 binding reagent complexes.

32. (canceled)

33. A computer-readable medium comprising code for controlling one or more processors to classify whether an ovarian or rectal cell and/or tissue sample from an subject is associated with ovarian or rectal cancer, said code comprising: instructions to apply a statistical process to a data set comprising a Galectin-7 profile to produce a statistically derived decision classifying said sample as an ovarian or rectal cancer sample or non-ovarian or rectal cancer sample based upon said Galectin-7 profile, wherein said Galectin-7 profile indicates the level of Galectin-7 in said ovarian or rectal cell and/or tissue sample.

34-37. (canceled)
